(19) United States
(12) Patent Application Publication
(10) Pub. No.: US 2005/0038450 A1
(43) Pub. Date: Feb. 17, 2005

(54) Methods and apparatus for minimally invasive transverse aortic banding

(76) Inventors: Ping Hu, Salt Lake City, UT (US); DongFang Zhang, Salt Lake City, UT (US); LeAnne Swenson, Salt Lake City, UT (US); Sheldon E. Litwin, Salt Lake City, UT (US)

Correspondence Address: TRASK BRITT, P.O. BOX 2550, SALT LAKE CITY, UT, 84110, US

(21) Appl. No.: 10/861,208

(22) Filed: Jun. 3, 2004

Related U.S. Application Data
(60) Provisional application No. 60/475,677, filed on Jun. 3, 2003.

(51) Int. Cl. A61B 17/10; A61B 17/04; A61L 17/00

(52) U.S. Cl. 606/148; 606/139; 606/228

(57) ABSTRACT

A curved suture applicator is provided for facilitation of ligation procedures in mammals. The said curved suture applicator enables a surgeon to efficiently encircle a target tissue such as a blood vessel with a suture or thread. A kit is also provided, comprising the said curved suture applicator, an appropriate length of suture or thread, and a suture manipulation device, such as a pair of forceps. Furthermore, a minimally invasive ligation method is provided that utilizes the said curved suture applicator and kit to efficiently band the transverse aorta of small mammals. Such banding is useful in studying and simulating the effects of left ventricular hypertrophy as it relates to congenital heart failure.

### CROSS REFERENCE TO RELATED APPLICATION

This application claims the benefit under 35 U.S.C. § 119(e) of U.S. Provisional Application No. 60/475,677, filed Jun. 3, 2003, which is incorporated herein by reference.

### GOVERNMENT RIGHTS

The United States government may have rights in the invention pursuant to grants from the Department of Veterans Affairs and the National Institute of Health (HL 52338-06, U01-70525, T32HL7576, HL5807).

### TECHNICAL FIELD

The present invention relates generally to biotechnology, more specifically, to ligation surgical techniques and a surgical apparatus. The present invention relates to minimally invasive methods and apparatus for vesicle constriction.

### BACKGROUND

Procedures for ligating tissues in surgical operations are frequently performed. Generally, ligation may take the form of a constricting band composed of a thread or suture. The thread or suture is passed around the target vessel and then tied into a constricting band. The knot-tying process may occur either inside (intracorporeal ligation) or outside (extracorporeal ligation) the body. Ligation of a vessel may also be achieved via use of a clip or staple.

Extracorporeal ligation is generally performed in the following manner. A thread (suture) is passed under a tissue of interest (i.e., a tissue for which the operational work is done). Then, both ends of the thread are drawn out of the subject's body and a knot is formed outside the body. The knot is put into the body by means of a thread feed instrument called a “knot pusher” or “knot driver,” which acts to tighten the knot. Generally, a square knot is used because it is not easily loosened.

In intracorporeal ligation, the knot is formed by using two forceps or other suture-holding devices inserted into the body cavity and the knot is formed inside the subject's body. Once again, a square knot is generally formed.

In both intra- and extracorporeal ligation, at least three primary difficulties must be overcome. First, the thread or suture must be passed around the vessel or tissue of interest. In certain surgeries, particularly in small animals, operational space may be very limited. Second, threading a suture around an object can require significant experience and/or be very time consuming. Again, if space is limited, tying the knot intracorporeally can be very challenging. Finally, ligation operations should not cause excessive damages to the regions being operated.

The ligation technique utilizing a clip or staple is advantageous in that the time-consuming knot-tying work required for extracorporeal ligation and intracorporeal ligation may be omitted. However, using this technique may require a more expensive apparatus for performing the ligation. Additionally, because foreign matter such as a clip may be left in the body, the range of application of this technique is limited.

With regards to intra- and extracorporeal ligation methods, several prior art devices are in existence to help overcome the challenges of passing and tying a thread around a tissue. However, many such devices do not allow the operator to pass a thread or suture around the tissue without first cutting through it. Furthermore, those devices that do not require initial cutting are generally complex, expensive, and usually require a large opening or port for inserting the device.

Such ligation devises are often used in the ligation or constriction of vessel-like membranes in mammals. One example of a procedure that involves ligation or constriction, is transverse aortic banding of small mammals, such as rats, mice, or guinea pigs. Constriction of the aorta has been used for many years to produce pressure-overload hypertrophy in small mammals, such as rodents, thus allowing researchers to study the causes of congenital heart failure. Generally, a constricting band is placed between the branches of the right and left carotid arteries. In order to access the aorta, the chest is entered in the second intercostal space at the left upper sternal border through a small incision. Then, aortic ligation is achieved by tying a nylon or silk suture ligature around the aorta and an accompanying blunt needle, which acts as a spacer to determine the scope of the constriction. When the needle is pulled out from within the suture loop, only the aorta remains within the constricting band. The result is that the aorta may be narrowed, for example, in mice the aorta is often constricted to about 0.4 mm in diameter, to produce a transverse aortic constriction (“TAC”) of 65-70%. However, one limitation to this particular example of a constriction protocol is that it causes a breach of the pleural space and necessitates mechanical ventilation of the mammal. The use of mechanical ventilation requires additional time, expertise and equipment. Moreover, inflammatory reactions within the chest may complicate the analyses of cardiac function and pathology.

In view of the foregoing discussion of ligation devices generally, and of ligation methods as applied to a tubular vessel, such as mammalian aortas, there is a need in the art for improved ligation devices and methods.

### SUMMARY OF THE INVENTION

The apparatus and/or methods of the invention may be applied to the constriction or ligation of a tubular vessel or other like-anatomical structure.

The invention provides an apparatus that is herein referred to as a curved suture applicator, whose purpose is to aid in the positioning of a thread or suture around a target tissue so that the thread or suture may be used to ligate or constrict the tissue.

The invention also provides a curved suture applicator comprising a hollow and/or solid tubular member. The distal end of the tubular member comprises a passage adequate for holding or passing a portion of a thread or suture. The tubular member comprises a straight portion and a bent or curved portion disposed at the distal end of the tubular member, thus allowing the distal end of the tubular member to be passed under the target tissue and thereby present an attached thread or suture in an accessible manner on the distal side of the target tissue.

The invention further provides a tubular member that may be covered by a soft flexible material or sheath, such as a plastic, rubber, Teflon® and other like materials which prevent or reduce abrasion of tissues within a subject. The invention further provides a soft flexible material that is slidably engaged to at least the tubular member.

The invention also provides a method for a minimally invasive ligation procedure, facilitating ligation or constriction of a vesicular membrane. When used in combination with a suture manipulation device such as a pair of forceps, the curved suture applicator is used to easily dispose a silk or nylon suture around a mammalian vesicle. Where appropriate, a predetermined constriction may be achieved by placing a spacing apparatus substantially parallel to the target vesicle. The suture is then tied, creating either a ligation or a constricting band around the vesicle.

An exemplary embodiment of the present invention is a transverse aortic banding (“MTAB”) procedure in mammals, utilizing the above-mentioned curved suture applicator. The MTAB method obviates the need for providing mechanical ventilation to the mammal because the pleural cavity of the mammal is not entered. Instead, access to the aorta is achieved by inserting the curved suture applicator through an incision in the mammal's sternum. When used in combination with a suture manipulation device such as a pair of forceps or a hooking devise, the curved suture applicator is used to easily dispose a suture around the mammalian aorta. A spacing apparatus, such as a blunt needle of a predetermined size is placed substantially parallel to the mammalian aorta and the suture is then tied, creating a constricting band around the aorta, thus inducing pressure-overload in the heart.

The present invention also includes a kit comprising a curved suture applicator that allow for the placement of a suture around an anatomical object. Optionally, the kit may contain one or more protective soft flexible materials that are adapted to cover at least the distal tip of the curved suture applicator. Optionally, the suture may be pre-loaded through the suture applicator. Optionally, the kit may contain additional suture. Optionally, suture kit may be sterilized via UV light or other methods known in the art. Optionally, the kit may contain an isolator consisting of rigid or semi-rigid member having a means for assisting in the isolation of tube to be ligated. In addition, the kit may be disposable.

### BRIEF DESCRIPTION OF THE DRAWINGS

FIG. 1 depicts a curved suture applicator with an optional soft flexible sheath covering the suture thread passage; the suture thread has been introduced into and is held by the distal end of the suture thread passage;

FIG. 2 depicts the curved suture applicator of FIG. 1, and demonstrates how the optional soft flexible sheath may be removed from the distal end of the suture thread passage in the hollow tubular member;

FIG. 3 depicts the curved suture applicator of FIG. 1, with the optional handle attached to the proximal end of the curved suture applicator;

FIG. 4 depicts a curved suture applicator with the optional soft flexible sheath covering the suture thread passage; the suture thread has been introduced into the distal end of the suture thread passage and threaded completely through the suture thread passage so that it exits the proximal end of the curved suture applicator;

FIG. 5 depicts a curved suture applicator whose distal end is positioned underneath a mammalian aorta; a suture thread has been threaded into the distal end of the suture thread passage, and has been positioned by the curved suture applicator so as to be easily accessed from above the aorta; and

FIG. 6 is a photograph of latex casts of aortas following MTAB (left) and sham surgery (right). The silk ligature has been removed from the MTAB aorta, but the site of narrowing is evident (arrow demonstrates location of constriction).

### DETAILED DESCRIPTION

The invention provides an apparatus and a method for constricting or ligating a vesicle or tissue in a subject. The curved suture applicator may be formed of a hollow and/or solid tubular member of hard material, which may optionally be covered by a softer material, such as plastic, rubber, silicon or Teflon®, to help protect the tissues within a subject. A suture thread passage for passing or holding a suture thread is formed at the distal end of the tubular member. In one exemplary embodiment, the inside diameter of a hollow tubular member is preferably greater than the outside diameter of the suture thread, which is to be inserted in the suture thread passage, thereby allowing the suture to be passed through at least the hollow tubular member.

The tubular member comprises a straight portion and a bent or curved portion disposed at the distal end of the tubular member. The bend or curvature of the tubular member is configured so that it may be passed under a vesicle and present the suture in an accessible manner.

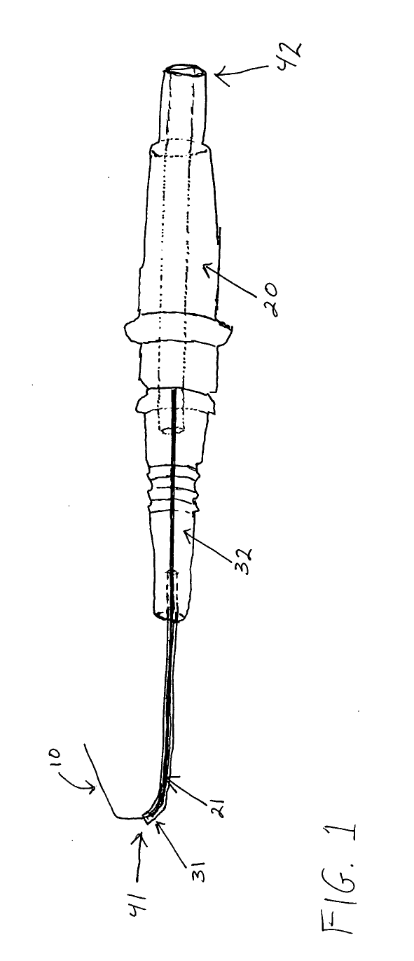

The device shown in FIG. 1 is an exemplary representation of a curved suture applicator. There, an angio-catheter 20, with distal end 41 and proximal end 42, was properly modified to include a curved, suture-thread passage 21 whose dimensions were adequate to position a suture around a mouse aorta. The suture thread passage 21 (a 24-gauge needle) was dimensioned at 24.5 mm in length and 0.7 mm in diameter. The silk suture 10 used was 6-0 suture thread (which may have a diameter between about 0.07 and about 0.099 mm), thus the silk suture may be inserted into the tip of the curved suture applicator, where it is held as the curved suture applicator was inserted into a subject. The tip of the applicator was bent at about a 50-degree angle, thus creating a curved suture applicator that could be positioned underneath the target tissue, allowing the held suture to be accessible from above the target tissue.

The bent portion of the applicator may be positioned relative to the distal tip 41 of the tubular member as is appropriate for the diameter of the target tissue. For example, the bend may be placed about 1.5 mm from the distal end of the tubular member. Finally, a soft plastic sheath 31 may be slidably engaged to the tubular member via the sheath connector 32 so as to avoid or reduce any accidental piercings or abrasions within the thoracic cavity during the procedure. In the case of the angio-catheter, the sheath was at least 25 mm in length. Thus, in this exemplary embodiment, the sheath covered the full length of the rigid hollow tubular member and was slidably engaged with the hollow tubular member, such that it could be extended from the curved end.

The exact dimensions shown in FIG. 1 are exemplary only. The dimensions of the curved suture applicator will vary according to the needs of the procedure. For example, it is envisioned that a curved suture applicator may be configured with two or three 50-degree bends in the tip of the tubular member. As will be apparent to a person of ordinary skill in the art, in light of the present disclosure, when two or more bends are utilized each bend may lie in the same plane or a different plane. Alternatively, one, two, or even three 30, 35, 40, 45, 50, 55, 60, 65 and/or 70 degree bends. For example, a single 70-degree bend in the tip of the tubular member is envisioned. Multiple bends of 10-, 20-, 40-, or 60-degrees, or any combination thereof, may be incorporated. The lengths of the unbent and bent tubular members may also vary according to the needs of the procedure. Finally, the diameter of a tubular member may vary, for instance, according to the diameter of the suture to be used in a given procedure. For example, when the suture is to be passed through the hollow tubular member, it is desirable that the hollow tubular member have a greater diameter than the diameter of the suture. Similarly, the diameter and length of the optional soft flexible sheath may change to accommodate the diameter and length of the tubular member.

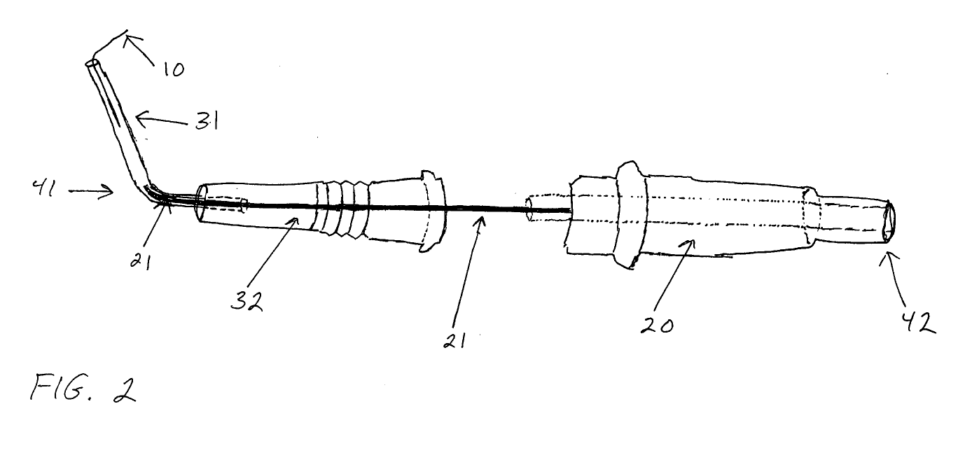

In another exemplary embodiment shown in FIG. 2, the suture 10 is fastened to the end of the suture thread passage 21. For example, the suture may be fastened to the end of a solid or semi solid tubular member by slidably engaging a flexible sheath 31 such that the suture is captured inside the tubular member or between the tubular member, which is encased in the sheath, and/or the suture may be passed through an aperture in the tubular member.

In an exemplary embodiment, the total length of the soft flexible sheath, if desired, may be longer than the exposed length of the tubular member. In another exemplary embodiment, a flexible sheath or covering may be shorter than the exposed length of the tubular member. In yet another exemplary embodiment, the flexible sheath or covering is affixed to the distal end (or tip) of the tubular member.

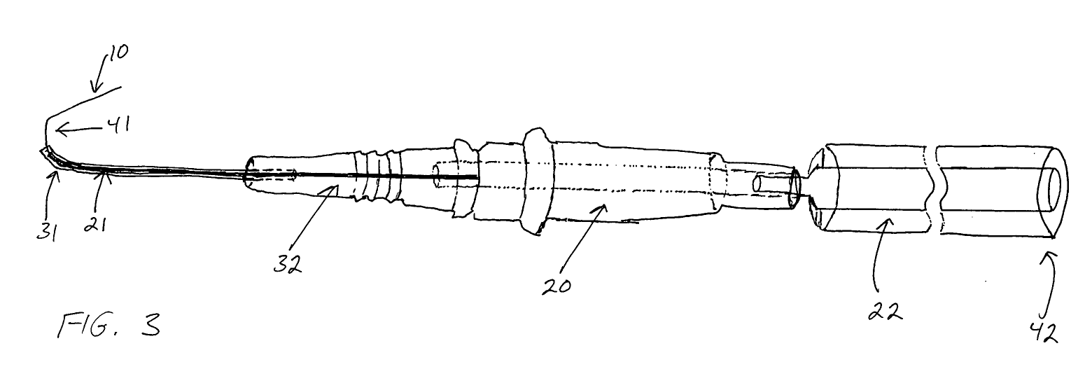

The tubular member may be configured to attach to a handle, as in FIG. 3. In one exemplary embodiment the tubular member 20 is attached to any of the connection devises known in the art (for example, a luer fittings, threaded connection means, glue, or the like), which facilitates the attachment of a handle or extension devise 22 as are known in the art. The handle or extension devise 22 may comprise an endoscopic surgical system

The tubular member may be made of a variety of hard materials. Various plastics or metals will suffice, preferably the material used may be sterilized and is non-reactive with organic material that it may come in contact with. The material used should be relatively stiff and able to “hold” its position or shape, even under moderate pressure. Similarly, the optional soft sheath may be made of various, inert and flexible materials such as plastic, rubber, silicon or Teflon®.

Methods and materials which may be useful in forming or constructing a tubular member, a flexible coating or sheath, retaining devises, and suture manipulation devises are described in U.S. Pat. Nos. 5,281,236; 5,702,407; 5,658,299; and 4,683,885, as well as International Patent Publications WO 04/004577 and WO 97/003615, which the entirety of each is hereby incorporated by reference.

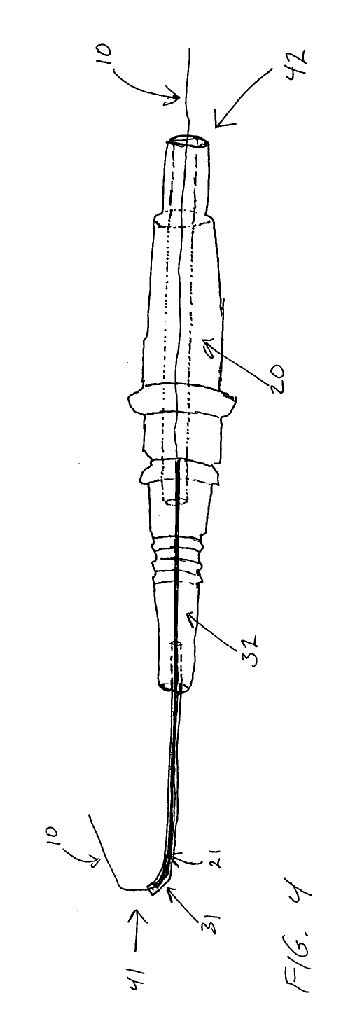

A second embodiment of the curved suture applicator, as shown in FIG. 4, comprises the same hollow tubular member 20 as previously outlined, but now further comprises an opening on the proximal end 42 of the tubular member. Thus, a suture thread 10 need not only be threaded into and held at the distal end 41 of the tubular member, but it may also be passed through the hollow tubular member 20 and secured at a site distal to the proximal end 42 of the hollow tubular member. For example, the suture may be secured in a retaining means on a handle affixed to the hollow tubular member, or outside of the patient's body. A length of suture thread would still protrude from the distal end of the hollow tubular member, thus allowing the curved suture applicator to be positioned in such a way as to make the distal end of the suture thread accessible for a ligation procedure. For example, a suture passed through the hollow tubular member may be retained by a retaining devise, such as a bobbin, hook, clasp or like structure.

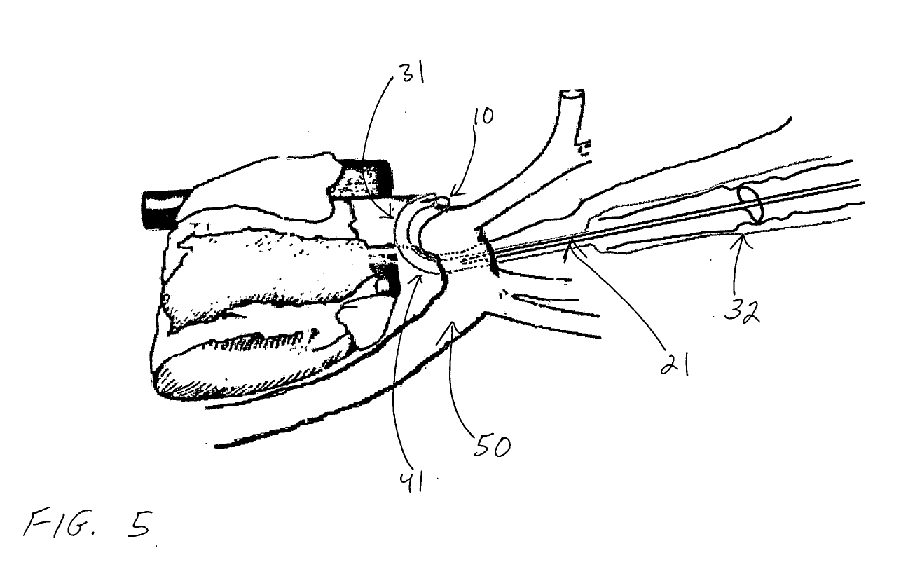

In an exemplary embodiment using the curved suture applicator, as demonstrated in FIG. 5, a minimally invasive technique for constricting a mammalian vesicle is provided. Upon accessing the vesicle 50, a length of surgical silk or nylon suture 10 should be applied to the vesicle 50. The distal end 41 of the curved suture applicator is inserted under the vesicle 50, thereby presenting the suture 10 on the distal side of the vesicle. A suture manipulation device, such as a pair of forceps, may be used to bring the suture back away from the vesicle 50. A spacer, such as a blunt 27-gauge needle, may then be placed next to the portion of the vesicle to be constricted, allowing the silk or nylon suture to be snugly tied about both the vesicle and the needle, either extra- or intracorporeally. The spacer may then be slid out from under the suture knot, leaving the vesicle constricted by the suture (e.g., a silk or nylon suture) to a diameter equal to that of the spacer used.

As will be apparent to a person of ordinary skill in the art after reading the disclosure, the spacer may be of any size or diameter appropriate to the desired constriction. Likewise, the spacer may be made of any material that does not deform under the pressure of the constricting suture. For example, the spacer may be metal or plastic.

The invention may include an isolator, which may be used to retract a tissue, organ, or vessel. An isolator may be a rigid or semi-rigid member having a hook, or other devise or means at one end, wherein the isolator facilitates the ability to grasp or engage a tissue, organ or vessel. The isolator may be covered by a soft material such as plastic, rubber or Teflon®, so as to further avoid the risk of accidental piercing of a tissue, organ or vessel within the body.

In another exemplary embodiment, a minimally invasive technique for constricting the transverse aorta in mice is provided that does not require mechanical ventilation because the pleural cavity is not breached (e.g., minimally invasive transverse aortic banding (MTAB)). The present invention can be used to consistently constrict the aorta in less than 10 minutes with rapid recovery and low morbidity and mortality. Not only is the approach of the present invention substantially faster and less expensive than previous methods, but potentially confounding experimental factors such as inflammation within the chest and healing of the chest wall incision are reduced. The invention produces changes in left ventricular size, function and expression of hypertrophic markers (i.e., increased expression of atrial natriuretic factor (ANF) and beta-myosin heavy chain) that are very comparable to those achieved with the conventional surgical technique. The use of a technique with lower mortality is particularly important, for example, when studying the effects of pressure-overload in rare or precious lines of genetically altered mice. The same ligation or constriction technique may be applied to other tissues such as the kidney, lung, stomach or muscle. Thus, the ligation or constriction technique may be used to survey changes in gene expression that occur in response (direct or indirect) to ligation or constriction of other vessels, tissues or organs. For example, a kidney or a part of a kidney of a subject may be constricted so as to facilitate the analysis of genes induced by such ligation, constriction or even kidney damage. Likewise, with other tissues, organs or vessels, such as the lung, stomach, muscle, etc.

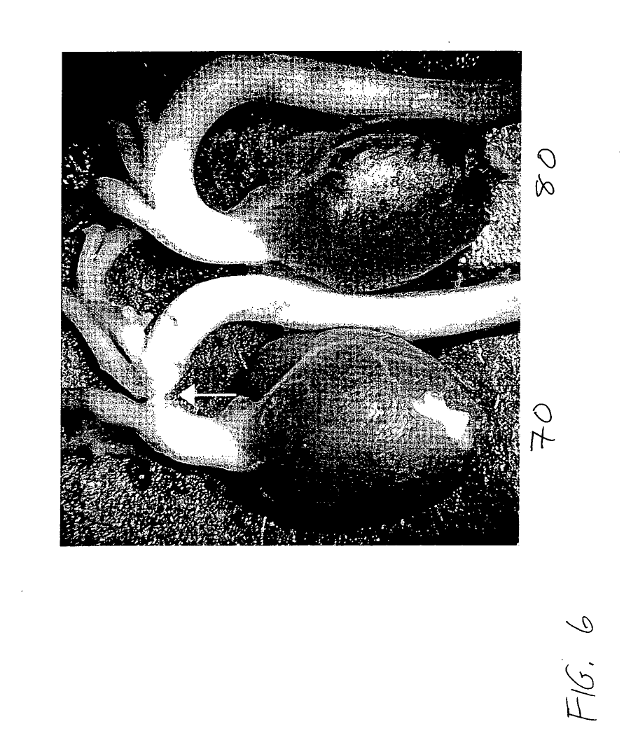

The MTAB technique, as embodied in the present invention, may be applied to small mammals, such as mice, rats, and guinea pigs. Prior to surgery, the experimental animal may, if appropriate and desirable, be anaesthetized. The initial step of the MTAB technique utilizes a 1.5-cm ventral midline incision, centered on the clavicle of the small mammal to be used in the experiment. Then, instead of accessing the aorta through an intercostal space, access is created by cutting a 2- to 3-mm longitudinal cut in the proximal portion of the sternum to the level of the second rib. Behind the sternum are the lobes of the thymus. The two lobes of the thymus should be divided, thus revealing the aortic arch as it crosses the trachea. The stretch of the aorta between the brachiocephalic and left carotid arteries should be isolated from surrounding tissue by utilizing a hook-like device that can effectively pull the aorta away from its neighboring tissue. Then a length of surgical suture is applied by inserting the curved suture applicator under and to the back of the aorta. A suture manipulation device, such as a pair of forceps, may be used to bring the suture back away from the aorta. A spacer, such as a blunt 27-gauge needle, may then be placed next to the isolated portion of the aorta, allowing the silk suture to be snugly tied about both the aorta and the needle. The spacer is then be slid out from under the suture knot, leaving the aorta constricted by the suture to a diameter equal to that of the spacer. Finally, the muscle and skin incisions may be closed using methods known in the art. The results of an application of the MTAB technique are demonstrated in FIG. 6. There, a heart with a constricted aorta 70 may be compared to a heart with an un-constricted aorta 80. The constriction in heart aorta 70 is indicated by the arrow in the figure.

The invention also provides a kit, which may be used to perform the MTAB procedure or to facilitate any other related tissue ligation or constriction procedure. Specifically, parts to be included in the kit will facilitate the placement and tying of a length of suture around an anatomical structure of a mammal. Such parts may include the above-described curved suture applicator, an appropriate length of suture (which may be either separate or pre-threaded in the applicator), a suture manipulation device, such as a pair of forceps or other clamping device, a spacer, a tool for isolating the vessel, and/or a set of instructions for proper use of the included curved suture applicator or protocol for performing the MTAB procedure.

The kit and/or the curved suture applicator may be used in a variety of surgical settings. The kit and/or curved suture presentation device may be used on any small vessel or nerve requiring constriction or ligation. Such needs occur in the aforementioned aortic surgery procedure, as well as in related pulmonary surgery. Similar uses may be found in reconstructive surgery, any type of microsurgery, craniofacial surgery, cosmetic surgery, and even hand surgery. Uses in gynecological and other reproductive surgery may be envisioned. Also, where appropriate and desirable, the kit and curved suture applicator may be used to apply a suture around muscle and/or tendons.

### EXAMPLE I

To further illustrate the present invention, consider once again the use of the curved suture applicator in the aforementioned MTAB technique. FIG. 1 is a photograph of latex casts of aortas following MTAB (left) and sham surgery (right). The silk ligature has been removed from the aorta, but the site of narrowing is evident (arrow demonstrates location of constriction). Aortic constriction, as shown in FIG. 1, and as performed by applying the present invention, may be used to artificially simulate hypertension, thus initiating a chain of events in the left ventricle of the heart in response to the higher blood pressure. Over a sufficient time period, the left ventricle of the affected heart will begin to enlarge. It is this resulting left ventricle hypertrophy that often leads to congenital heart failure. Thus, by using the present invention to simulate left ventricular hypertrophy, other studies and experiments may be conducted on the hypertrophied heart to determine the nature and pathology of the responsible gene signaling pathways or a signal transduction cascade. Once identified, treatments for congenital heart disease, for example, treatments that act to block the signaling pathways or cascades causing left ventricular hypertrophy, may be developed.

Although the present invention has been described with respect to the illustrated embodiments, various additions, deletions and modifications are contemplated as being within the scope of the invention. The scope of the invention is, therefore, indicated by the ensuing claims, rather than the foregoing description. All changes that come within the meaning and range of equivalency of the claims are to be embraced within their scope.

All references presented herein using open terminology such as, comprising and/or including, contemplates closed terminology, such as consisting of and consisting essentially of.

All references, including publications, patents, and patent applications, cited herein are hereby incorporated by reference to the same extent as if each reference were individually and specifically indicated to be incorporated by reference and were set forth in its entirety herein.

1) A curved suture applicator, comprising a handle and a suture holding tubular member curved to facilitate presentation of a length of suture around an anatomical structure in an animal.

2) The curved suture applicator of claim 1, wherein the suture holding tubular member is encased by a soft flexible sheath.

3) The curved suture applicator of claim 2, wherein the soft flexible sheath is longer than the suture holding element.

4) The curved suture applicator of claim 2, wherein the soft flexible sheath is shorter than the suture holding element.

5) The curved suture applicator of claim 1, wherein the suture holding tubular member is a hollow tubular member.

6) The suture holding element of claim 4, wherein the suture holding tubular member further comprises a 10 to 130 degree curve.

7) The curved suture applicator of claim 4, wherein the suture holding tubular member further comprises a 50 degree curve.

8) The curved suture applicator of claim 4, wherein the suture holding tubular member further comprises a 30 degree curve.

9) The curved suture applicator of claim 4, wherein the suture holding tubular member further comprises two 30 degree curves.

10) The curved suture applicator of claim 4, wherein the suture holding tubular member further comprises three 30 degree curves.

11) The curved suture applicator of claim 4, wherein the suture holding tubular member further comprises a 70 degree curve.

12) A curved suture applicator, consisting of:
a suture holding hollow tubular member having a proximal end and a distal end, wherein the distal end is configured to receive a length of a suture;
a 50 degree curve proximal to the distal end to facilitate presentation of the length of suture around an anatomical structure in an animal; and
a soft plastic sheath encasing the suture holding hollow tubular member.

13) The curved suture applicator of claim 12, wherein the soft flexible sheath is longer than the suture holding hollow tubular member.

14) The suture holding tubular member of claim 13, wherein the soft flexible sheath is slidably engaged to the suture holding hollow tubular structure.

15) A kit for performing a minimally invasive ligation or constriction procedure, the kit comprising:
the curved suture applicator of claim 4; and
a length of suture.

16) The kit of claim 15, further comprising a suture manipulation device.

17) The kit of claim 15, further comprising instructions.

18) The kit of claim 15, further comprising a spacer.

19) The kit of claim 15, further comprising an isolator tool.

20) The kit of claim 15, wherein the curved suture applicator comprises a 50 degree curve.

21) A kit for performing a minimally invasive ligation or constriction procedure, the kit consisting of:
the curved suture applicator of claim 12;
a length of suture;
a suture manipulation device; and
instructions.

22) A minimally invasive ligation method for disposing a length of suture around an anatomical structure of an animal, comprising:
inserting the curved suture applicator of claim 4 into the body of an animal, wherein the suture holding tubular member carries a length of suture through an incision in the animal;
passing the curved applicator having the suture under an anatomical structure in the animal;
manipulating the suture so as to encircle the anatomical structure of the animal;
disposing a spacer proximate to the anatomical structure;
tying the encircling length of suture around the anatomical structure and the spacer; and
removing the spacer.

23) The minimally invasive method of claim 22, wherein the animal is a mammal.

24) The minimally invasive method of claim 23, wherein the anatomical structure to be encircled is an aorta, and the mammal is a small mammal.

25) The minimally invasive method of claim 24, wherein inserting the curved suture applicator does not breach the mammal's pleural space.

26) The minimally invasive method of claim 25, further comprising forming a small incision in the mammal's sternum and inserting the curved suture applicator through the incision in the small mammal's sternum.

27) The minimally invasive method of claim 26, wherein the small mammal is a mouse.

28) The minimally invasive method of claim 23, wherein the small mammal is a mouse.
